(19) United States
(12) Patent Application Publication
(10) Pub. No.: US 2009/0131959 A1
(43) Pub. Date: May 21, 2009

(54) Biological Vessel Flow Control Devices and Methods

(75) Inventor: Jason P. Rolland, Durham, NC (US)

Correspondence Address: MORGAN, LEWIS & BOCKIUS LLP, 1701 MARKET STREET, PHILADELPHIA, PA, 19103-2921, US

(73) Assignee: Liquidia Technologies Inc., Research Triangle Park, NC (US)

(21) Appl. No.: 12/297,516

(22) PCT Filed: Apr. 20, 2007
(86) PCT No.: PCT/US07/09792

§ 371 Date: Oct. 17, 2008

Related U.S. Application Data
(60) Provisional application No. 60/745,238, filed on Apr. 20, 2006.

(51) Int. Cl. A61B 17/122; A61F 2/04; A61F 6/20

(52) U.S. Cl. 606/158; 600/30; 606/157; 128/831; 128/843

(57) ABSTRACT

A medical device including a body portion configured and dimensioned to be associated with a vessel of a patient and a responsive component associated with the body portion wherein the responsive component is switchable between a first configuration and a second configuration.

### RELATED APPLICATIONS

This application claims priority to U.S. Provisional Patent Application 60/745,238 which was filed Apr. 20, 2006 and which is hereby incorporated by reference in its entirety.

### INCORPORATION BY REFERENCE

All references cited herein are hereby incorporated by reference as if set forth in their entirety herewith.

### FIELD OF THE INVENTION

Generally, the present invention is related to implantable biomedical devices. More particularly the implantable devices include valves for controlling flow in a vessel or duct.

### BACKGROUND OF THE INVENTION

Contraception methods can be broken down into three categories: chemical, mechanical, and surgical. Chemical contraception, used almost exclusively by the female population, takes the form of a pill, implant, or patch which is used to deliver hormones or drugs to prevent ovulation. While chemical contraception has proven effective, there are concerns among the general population about its safety.

Non-chemical methods of contraception, or “mechanical” contraception methods, are also known. These generally employ physical methods that prevent sperm or ova from reaching target areas in the body. Examples of mechanical contraception include condoms, diaphragms, and other devices. Mechanical contraception is generally less effective than chemical contraception and may lead to discomfort.

Finally, there are surgical methods of contraception. The most common among these are vasectomy in men (where the vas deferens are cut) and tubal ligation in females (where the fallopian tubes are closed).

Advances in male contraception have been lagging behind those made in female contraception. Surgical sterilization, such as vasectomies (referred to herein as “first generation” techniques) possess intrinsic disadvantages in that they are reversible only through complicated, low success surgeries.

Newer techniques have emerged which result in less permanent damage to the vas deferens (referred to herein as “second generation” techniques). These techniques generally employ specialized devices or implants. While such implants have shown success, they still require invasive surgery for reversal.

Finally, there exist certain polyelectrolyte gels which exhibit contraceptive ability in males. Such methods are temporary as the materials degrade over time, or they can be removed through invasive solvent washes.

Thus, there exists a clear need for the next generation male contraceptive device with is non-invasively reversible at an individual's request.

### SUMMARY OF THE PREFERRED EMBODIMENTS

According to some embodiments, a medical device includes a body portion configured and dimensioned to be associated with a vessel of a patient; and a responsive component associated with the body portion where the responsive component is switchable between a first configuration and a second configuration.

In some embodiments, the first configuration restricts flow through the vessel and the second configuration does not restrict flow through the vessel. In some embodiments, the medical device includes a controller configured to manipulate the responsive component between the first configuration and the second configuration. In some embodiments, the medical device includes an indicator operatively associated with the responsive component for indicating whether the responsive component is in the first configuration or the second configuration.

In some embodiments, the responsive component is an electrostrictive, magnetostrictive or piezoelectric actuator, a shape-memory polymer, or at least one ferromagnetic particle.

In some embodiments, the body portion is configured and dimensioned to receive an external surface of the vessel or to be positioned within the vessel. According to some embodiments, the body portion is substantially cylindrical in shape, and the body portion has an elongate slit formed axially therein through which the vessel may pass. In some embodiments, the vessel is a vas deferens, a fallopian tube, a urethra, a ureter, a duct, intestine, an artery, and/or a vein. In some embodiments, the body portion includes a material that inhibits tissue growth or adhesion of substances.

According to certain embodiments, a medical device includes a body portion configured and dimensioned to be positioned in a vessel of a subject without cutting through the vessel; a channel defined in the body portion through which fluid in the vessel may pass; and a responsive component associated with the body portion for reversibly closing the channel such that fluid in the vessel is restricted from flowing through the channel.

In some embodiments, a medical device includes a channel defined in the body portion, where the channel has an inner diameter of not more than about 5 centimeters; not more than about 3 centimeters; not more than about 2 centimeters; not more than about 1.5 centimeters; not more than about 1 centimeter; not more than about 8 millimeters; not more than about 6 millimeters; not more than about 4 millimeters; not more than about 2 millimeters; not more than about 1.5 millimeters; not more than about 1 millimeter; not more than about 0.5 millimeters; not more than about 0.25 millimeters; not more than about 0.2 millimeters; not more than about 0.1 millimeters; or not more than about 0.05 millimeters.

In some embodiments, the outer diameter of the body portion is less than about one centimeter; less than about 5 millimeters; less than about 2.5 millimeters; less than about 1.5 millimeters; less than about one millimeter; less than about 0.8 millimeters; less than about 0.6 millimeters; or less than about 0.5 millimeters.

One embodiment of the present invention also includes methods for controlling flow through a vessel of a patient. According to some embodiments, a method of controlling flow through a vessel of a patient includes associating a medical device with a vessel of a patient where the medical device includes a body portion configured and dimensioned to be associated with the vessel of the patient; a responsive component associated with the body portion; and controlling the responsive component to restrict or allow flow through the vessel. In some embodiments, the medical device is implanted within the vessel. In other embodiments, the body portion is associated with an outer diameter of the vessel.

A further embodiment of the invention is a combination comprising an implantable medical device, particularly configured and dimensioned for insertion within a vessel, operatively associated with a guide wire or tube. The medical device and guide wire or tube may be configured for guiding the medical device into a vessel for insertion at a desired location. Medical devices, or combinations of medical devices and guide wires or guide tubes, can be packaged together in a container in sterile form for subsequent use.

Reference is made to the accompanying drawings in which are drawn illustrative embodiments of the invention.

### BRIEF DESCRIPTION OF THE DRAWINGS

FIGS. 1a-1d show a schematic illustration of a process for fabricating devices according to embodiments of the present invention;

FIGS. 2a and 2b show an implantable medical device containing a reversible valve component; FIG. 2a illustrates a device in an open position and FIG. 2b illustrates a devices in a closed position according to embodiments of the present invention;

FIG. 3 shows a semicircular device containing active components according to embodiments of the present invention;

FIGS. 4a and 4b are cross sectional views of a moveable valve inside a device according to embodiments of the present invention;

FIGS. 5a and 5b are a representation of moveable check valves that can be operated by an applied external or internal stimulus according to embodiments of the present invention; and

FIGS. 6a and 6b are a representation of a device design that allows selective bypass of the valve according to an embodiment of the present invention.

FIG. 7 is a representation of master template with a channel and check valve structure.

### DETAILED DESCRIPTION OF PREFERRED EMBODIMENTS

The foregoing and other aspects of the present invention will now be described in more detail with respect to the embodiments described herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art. The disclosures of all United States patent references cited herein are to be incorporated by reference herein in their entirety.

“Vessel” as used herein may be any duct, vessel, tube, or the like within a subject, including but not limited to arteries, veins (e.g., in the treatment of varicose veins, circle of willis, or the like), vas deferens (including the ejaculatory ducts, e.g., as a means of birth control), ureters (e.g., for the treatment of vesicoureteral reflux), uterine tubes or fallopian tubes (e.g., as a means of birth control), urethra (e.g., for the treatment of incontinence), ducts of glands (including but not limited to exocrine glands, lacrimal or tear glands, salivary glands, the pancreas, mammary gland, adrenal glands, pituitary glands, etc.), air passages (e.g., bronchi, etc.), esophagus, intestine (e.g., small intestine, large intestine, bowl, etc.), and the like.

“Normally open” as used herein refers to an object that maintains an open configuration in the absence of application of an external stimulus or signal. For example, a valve positioned on or in a ureter is preferably a normally open valve so that the potential for backup of urine into the kidney is minimized.

“Normally closed” as used herein refers to an object that maintains a closed configuration in the absence of application of an external stimulus or signal. For example, a valve positioned on or in a urethra is preferably a normally closed valve so that the potential for an episode of incontinence is minimized.

“Switchable” as used herein refers to an object that maintains either an open or closed position until a first external stimulus or signal is applied. For example, upon application of a first external stimulus or signal, the valve changes or switches to the opposite position and maintains that opposite position when the stimulus or signal is removed. The valve returns to the original position when a second stimulus or signal is applied, and then maintains that original position when the second stimulus or signal is removed. For example, a valve of the present invention positioned on or in a fallopian tube or vas deferens may, in some embodiments, be a switchable valve.

Responsive components as used herein may include any suitable actuator, including but not limited to mechanical, piezoelectric, electroconstrictive, magnetostrictive actuators, combinations thereof, and the like. See, e.g., U.S. Pat. Nos. 6,946,097; 6,924,589; 6,686,882; and 6,526,864, each of which is incorporated herein by reference. In some embodiments, the responsive component can include “micromuscles” as described in U.S. Pat. No. 6,933,659, which is incorporated herein by reference. Such responsive component may be configured in any suitable manner to provide a normally open, normally closed, or switchable valve as described above. In some embodiments, a responsive component may employ the application of energy such as an electrical or magnetic field or the like. In some embodiments, energy may be applied to a responsive component from an external controller and/or from an internal controller (e.g., an electromagnet operatively associated with the body portion). In some embodiments, an internal controller may be energized by operative association with an antenna also implanted into the subject, which antenna may receive energy from an external controller. See, e.g., U.S. Pat. Nos. 6,308,101; 5,697,951; and 4,524,774, each of which is incorporated herein by reference.

Indicator as used herein includes both active (e.g., emitting a signal) and passive (e.g., detectable upon application of an external signal) indicators. Examples include but are not limited to contrast agents incorporated into the device (e.g., a stationary reference contrast agent and a contrast agent incorporated into the responsive component or valve, or a segment of the body portion, that moves in relationship to the reference agent), RFIDs, sensors and transmitters, including but not limited to that described in U.S. Pat. Nos. 6,009,350; 6,847,844; and 6,580,948, each of which is incorporated herein by reference. In some embodiments, the device may be operatively associated with an external receiver for providing audible feedback to the patient from the indicator, such as described in U.S. Pat. No. 5,009,644, incorporated herein by reference, to indicate a desired (or undesired) valve position. In some embodiments, the indicator may be configured to provide information to an external receiver positioned close to the patient for transmission to a remote location, as described in U.S. Pat. No. 6,805,667, which is incorporated herein by reference.

“Shape memory polymers” are known and described in, for example, U.S. Pat. No. 6,720,402 to Langer et al., which is incorporated herein by reference. In some embodiments, shape memory polymers can be natural or synthetic, and thermoset or thermoplastic. The polymer may be in any form, such as a graft polymers linear polymer, dendrimer polymers, combinations thereof, and the like. In some embodiments, the polymer may be a composition that includes: (a) at least one hard segment (e.g., which hard segment has a Tₜᵣₐₙₛ between about −40 and about 270° C.), (b) a first soft segment (e.g., which first soft segment has a Tₜᵣₐₙₛ at least about 10° C. lower than that of the hard segment(s)), which is linked to at least one hard segment, and (c) a second soft segment, linked to at least one of the hard segment or first soft segment (e.g., which second soft segment has a Tₜᵣₐₙₛ at least about 10° C. less than the Tₜᵣₐₙₛs of the first soft segment). The polymer may include multiple segments. In some embodiments, the molecular weight Mₙ of at least one of the segments can be between about 500 and about 10,000. Such shape memory polymers may be formed into or include valves or responsive components controlled by any suitable technique, such as by incorporation of nanoparticles or magnetoparticles therein for heating. See, e.g., R. Mohr et al., Initiation of shape-memory effect by inductive heating of magnetic nanoparticles in thermoplastic polymers, Proc. Natl. Acad. Sci. 103, 3540-3545 (Mar. 7, 2006), which is incorporated herein by reference.

“Soft lithography” includes fabrication procedures utilizing elastomeric stamps, molds, and/or conformable photomasks. Examples include microcontact printing, replica molding, microtransfer molding, micromolding in capillaries, solvent-assisted micromolding, etc. Soft lithography processes are known and can be found in U.S. Pat. Nos. 7,000,684; 6,988,534; 6,975,765; 6,952,436; 6,794,196; 6,663,820; 6,586,885; and 6,521,489, each of which is incorporated herein by reference.

Subjects that may be implanted with or treated with the devices or methods described herein include human subjects (including both males and females), as well as animal subjects (including but not limited to mammals such as dogs, cats, horses, sheep, cattle, monkeys, baboons, etc.) for veterinary medical purposes.

Some embodiments include a contraceptive implantable device containing a reversible switch or valve that can control the flow of spermatozoa cells or ova through a given channel. In some embodiments, the device can take the shape of a round tube which contains a channel or channels. The channel or channels may be reversibly closed using a valve or switch associated with or embedded within the device. In some embodiments, the device can be designed such that it fits inside of a vessel, such as but not limited to the vas deferentia in men or the fallopian tubes in women. Alternatively, the device can be designed such that it fits on or manipulates the outside of a vessel, such as but not limited to the vas deferentia in men or the fallopian tubes in women. In some embodiments the device of the present invention is used as a contraceptive tool in non-human mammals or animals.

Typical inner diameters of vasa deferentia and fallopian tubes are on the order of millimeters. Devices which control fluids at this size scale are often produced by microfabrication techniques such as those used to fabricate microfluidic devices. Microfluidic devices have emerged as a powerful technology for the manipulation of fluids at small volumes, as described in Science 2000 290: 1536-1540, which is incorporated herein by reference. Microfluidic devices typically contain channels on the order of 50 to 100 microns in width. Micro-scale features within medical and microfluidic devices can be fabricated by a number of methods including lithography, injection molding, and so called “soft lithography” techniques, which is described in Angewandte Chemie International Edition Volume 37, Issue 5, 550-575, and incorporated herein by reference. In some embodiments, soft lithographic methods similar to those employed to produce microfluidic chips are used to fabricate the devices of the present invention.

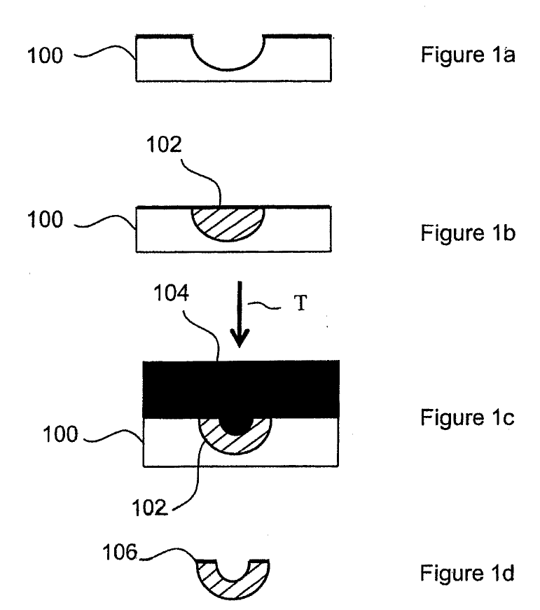

Referring now to FIGS. 1a-1d, a lithography process is shown as a method for fabricating the devices of the present invention. The lithography process includes providing master template 100 which includes a desired pattern, as shown in FIG. 1a. The desired pattern of master template 100 can be formed using traditional photolithography techniques which are well known in the art. Next, in FIGS. 1b and 1c, a liquid material 102 is introduced to template 100 and treated, as indicated by arrow T, to cured or solidified liquid material 102 into solid device 106 which retains the shape characteristics of master template 100. In some embodiments, a second mold 104 can be introduced to give more complex shapes or characteristics to device 106. In some embodiments, liquid material 102 is a silicone rubber precursor such as that sold by Dow Corning under the trade name SYLGARD 184™. In some embodiments, liquid material 102 is cured or hardened by treating liquid material with treatment T. Treatment T can be photo-curing, actinic radiation, thermal curing, evaporation, combinations thereof, or the like. Solid device 106 is removed from master template 100 and retains a pattern with a negative image of master template 100. In some embodiments, complex devices can be formed by fabricating multiple patterned solid devices 106 and coupling such multiple devices together in a predetermined organization. Multiple solid devices 106 can be coupled by known techniques in the art such as techniques described in Quake, et. al. Science 2000 288: 113-116, which is incorporated herein by reference. Other useful methods and materials for fabricating the devices of the present invention are disclosed in PCT Patent Application No. PCT/US06/23722, and PCT/US06/31067, which are incorporated herein by reference.

At the heart of microfluidics is the ability to control fluid flow. To this end, a number of valve technologies have been described which allow for such control over fluidic flow. Of particular relevance to this invention are valves designed for use in microfluidic chips made from soft materials such as silicones. Such valve designs often have so called “diaphragm valves” which are actuated by external stimuli.

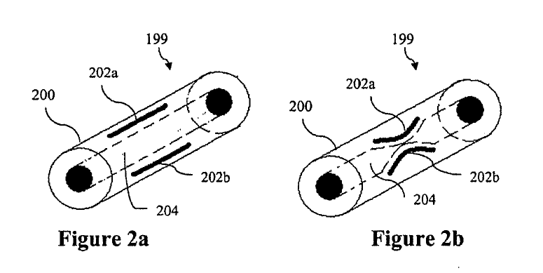

Referring to FIGS. 2a-2b, device 199 of the present invention may include a body portion 200, at least one channel 204 in body portion 200, and responsive component 202a, 202b operatively associated with body portion 200 and channel 204 for opening or closing channel 204. In some embodiments, body portion 200 can include single responsive component 202a, or multiple responsive components 202a, 202b which can form a valve. In some embodiments, body portion 200 may be configured for positioning around a vessel. In some embodiments, body portion 200 may be configured for positioning in a vessel. In some embodiments, body portion 200 can be configured for positioning near or in communication with a vessel such as to manipulate or control flow through the vessel. Preferably, body portion 200 may be configured for positioning around or in the vessel without cutting through the vessel or otherwise disturbing the tissue of the natural vessel.

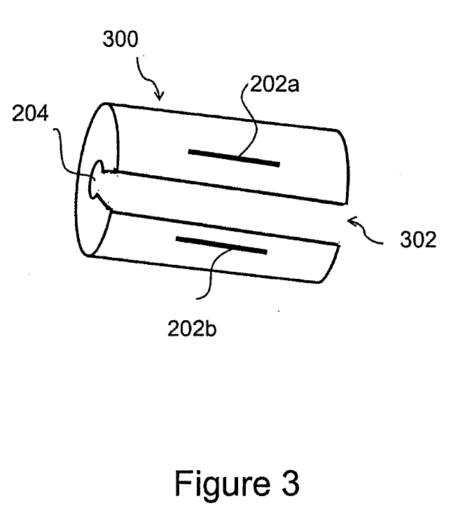

In some embodiments, body portion 200 is cylindrical in shape with channel 204 formed therein. In further embodiments, body portion 200 may have an elongate slit 304 (FIG. 3) formed along an axis of body portion 200 through which a vessel may pass into channel 204.

In some embodiments, body portion 200 of device 199 of the present invention can have an outer diameter of less than about 1 mm, 3 mm, 5 mm, 10 mm, 1.5 cm, 2 cm, 2.5 cm, 3 cm, 3.5 cm, 4 cm, 4.5 cm, 5 cm, or 10 cm. In some embodiments, a channel in device 199 can have an inner diameter of not more than about 0.05 mm. In some embodiments, a channel in device 199 can have an inner diameter of not more than about 0.1 mm. In some embodiments, a channel in device 199 can have an inner diameter of not more than about 0.15 mm. In some embodiments, a channel in device 199 can have an inner diameter of not more than about 0.2 mm. In some embodiments, a channel in device 199 can have an inner diameter of not more than about 0.25 mm. In some embodiments, a channel in device 199 can have an inner diameter of not more than about 0.5 mm. In some embodiments, a channel in device 199 can have an inner diameter of not more than about 0.6 mm. In some embodiments, a channel in device 199 can have an inner diameter of not more than about 0.8 mm. In some embodiments, a channel in device 199 can have an inner diameter of not more than about 1 mm. In some embodiments, a channel in device 199 can have an inner diameter of not more than about 1.5 mm. In some embodiments, a channel in device 199 can have an inner diameter of not more than about 2 mm. In some embodiments, a channel in device 199 can have an inner diameter of not more than about 2.5 mm. In some embodiments, a channel in device 199 can have an inner diameter of not more than about 3 mm. In some embodiments, a channel in device 199 can have an inner diameter of not more than about 4 mm. In some embodiments, a channel in device 199 can have an inner diameter of not more than about 5 mm. In some embodiments, a channel in device 199 can have an inner diameter of not more than about 6 mm. In some embodiments, a channel in device 199 can have an inner diameter of not more than about 7 mm. In some embodiments, a channel in device 199 can have an inner diameter of not more than about 8 mm. In some embodiments, a channel in device 199 can have an inner diameter of not more than about 9 mm. In some embodiments, a channel in device 199 can have an inner diameter of not more than about 1 cm. In some embodiments, a channel in device 199 can have an inner diameter of not more than about 1.5 cm. In some embodiments, a channel in device 199 can have an inner diameter of not more than about 2 cm. In some embodiments, a channel in device 199 can have an inner diameter of not more than about 2.5 cm. In some embodiments, a channel in device 199 can have an inner diameter of not more than about 3 cm. In some embodiments, a channel in device 199 can have an inner diameter of not more than about 3.5 mm. In some embodiments, a channel in device 199 can have an inner diameter of not more than about 4 cm. In some embodiments, a channel in device 199 can have an inner diameter of not more than about 5 cm.

In some embodiments, the devices in the present invention may be fabricated using soft lithographic methods as described herein and in the documents incorporated herein by reference. In some embodiments, fabrication of the devices may be accomplished by pouring liquid precursor material into molds of the desired shape and curing the liquid precursor material such that the cured liquid material retains the shape of the mold. In some embodiments the devices are hollow tubes, as shown in FIGS. 2A and 2B. According to such embodiments, devices 199 can contain responsive components 202a and 202b. Responsive components 202a and 202b may be components that are capable of being stimulated by an external and/or internal stimulus generated from a controller to reconfigure into a different configuration. In some embodiments, responsive components 202A and 202B can be configured to change a configuration to open (FIG. 2a) or close (FIG. 2b) channel 204 in response to an external and/or internal stimulus. In other embodiments, the external and/or internal stimulus can be a magnetic field stimulus, radio frequency stimulus, weak electrical fields, light waves, changes in temperature, ultrasound, radiation, X-Rays, physical manipulation, combinations thereof, or the like.

Referring now to FIG. 3, a device of the present invention may be semicircular device 300. In some embodiments, semicircular device 300 may be fabricated with a longitudinal opening 302 and an inner channel 204. Opening 302 can be configured to be a length wise or axial opening or slit along semicircular device 300. Opening 302 can be utilized for positioning semicircular device 300 over a vessel such that the vessel is housed in channel 204. Semicircular device 300 can then be fixed on the outside of vessel 304. In some embodiments, vessel 304 may be, but is not limited to, a vas deferens, a gland duct, a blood vessel, bronchi, intestine, or the like. Semicircular device 300 may contain responsive components 202a and 202b. In some embodiments, responsive components 202a and 202b may be stimulated by an external controller to open or close vessel 304. Responsive components 202a, 202b can include a reversible valve or switch that can be activated non-invasively by, but not limited to: magnetic fields, weak electrical fields, light waves, changes in temperature, ultrasound, radiation, X-Rays, physical manipulation, combinations thereof, or the like. In some embodiments, responsive components 202a, 202b include magnetic materials that can be magnetized and demagnetized reversibly, thereby forming a switch that closes and opens channel 204. In further embodiments, the device or parts of the device are made of silicone rubber that may be doped with iron oxide particles or magnetite. In some embodiments, these magnetic materials are so called “ferrofluids.” In other embodiments the device contains metals or metal alloys which display attraction to magnetic materials.

In some embodiments responsive components 202a, 202b include a simple metal shape (e.g., a wire, rod, or the like) embedded in the body of device 199, 300 that can be bent and hold a given position to close a valve. In other embodiments responsive components 202a, 202b include polymers with so-called “shape memory.” The shape of a polymer in one shape memory embodiment can be changed by activation with heat, light, combinations thereof, or the like. Such materials are generally known and described in U.S. Pat. No. 6,720,402, and Science 2002 296: 1673-1676, each of which are incorporated herein in their entirety. In still other embodiments, responsive components 202a, 202b of the device include magnetic shape memory materials. Other materials and techniques for forming responsive components 202a, 202b include, but are not limited to, piezoelectric materials and other materials known in the art.

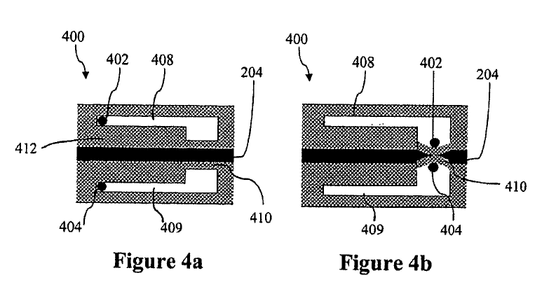

An example of a valve activated by magnetic fields for controlling flow in a vessel according to the present invention is shown as a cross section in FIGS. 4a and 4b. According to this design, device 400 includes channel 204 for receiving a vessel. Device 400 includes magnetic bead or particle 402 placed within first feature 408. Second magnetic or metal bead or particle 404 is placed opposite first magnetic bead or particle 402 and in second feature 409. Magnetic beads or particles 402 and 404 are configured to be attracted to one another and manipulated from an internal or external controller. In some embodiments, magnetic beads or particles 402 and 404 can be manipulated externally with a magnetic field to move up and down first feature 408 and second feature 409, respectively, within device 400. When beads 402 and 404 are in a first position 412 in the device with membrane 410 separating particles or magnetic/metal beads 402 and 404 is of a thickness, stiffness, or the like that maintains particles 402 and 404 from interacting. In some embodiments, first position 412 that maintains particles 402 and 404 from interacting can include a predetermined distance between particles 402 and 404. A second position 410 of feature 408 includes a position where magnetic/metal beads or particles 402 and 404 can interact and thereby close channel 204. In some embodiments, material of device 400 can be of a certain thickness at second position 410, such that it collapses, thus sealing channel 204. To open channel 204, magnetic/metal beads or particles 402 and 404 may be moved to first position 412 of device 400 where membrane of the device separating particles 402 and 404 is of a thickness that the attraction of particles 402 and 404 is not sufficient to collapse, thus opening channel 204. Thus, magnetic beads or particles 402 and 404 can be moved back and forth between first position 412 and second position 410 using an external or internal magnetic field and opening and closing channel 406.

In further embodiments a static magnetic component can be embedded into the silicone rubber and is manipulated in a similar manner with a moving magnetic component. A device containing multiple layers of such features can be fabricated by multi-layer soft lithography or other methods, as described herein. The manipulation of such responsive components or valves can be monitored with the use of ultrasound imaging etc.

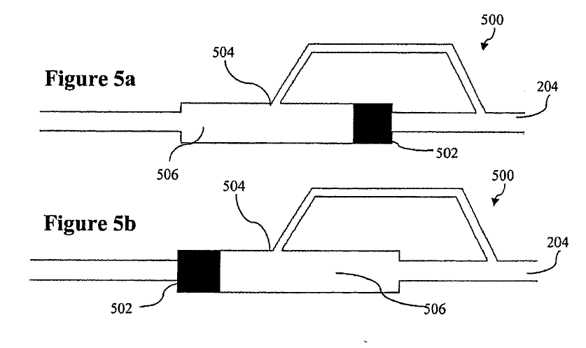

Referring now to FIGS. 5a and 5b, device 500 contains patterns that include check valves. In some embodiments, channels 204 may be patterned into device 500 such that movable plug valve 504 can be configured within valve region of channel 506. In some embodiments, plug 502 of movable plug valve 504 can include a metal bead or particle or a photopolymerized polymer as described in Anal Chem. 2002, 74, 4913, which is incorporated herein by reference. In further embodiments, the photopolymerized polymer contains magnetic particles. In some embodiments, plug 502 may be formed by introducing the prepolymerized fluid into device 500 and curing it in the selected area either by masking the rest of valve region of channel 506 or by only introducing a desired volume of liquid into valve region of channel 506. In further embodiments, plug 502 is formed of a viscous magnetic fluid and controlled by applied magnetic forces. In some embodiments, device 500 contains magnets or metal elements on both ends of valve region of channel 506 that allows for magnetic attraction of plug 502 to one end or the other of valve region of channel 506. As shown in FIG. 5a, when plug 502 is moved to a first position within valve region of channel 506, channel 204 is open and fluid or substance can flow across magnetic plug valve 504. Conversely, as shown in FIG. 5b, when plug 502 is moved to a second position within valve region of channel 506, channel 204 is closed and fluid or substance is restricted or blocked from flowing across magnetic plug valve 504. In some embodiments, plug 502 is moved back and forth in channel 506 using an external magnet.

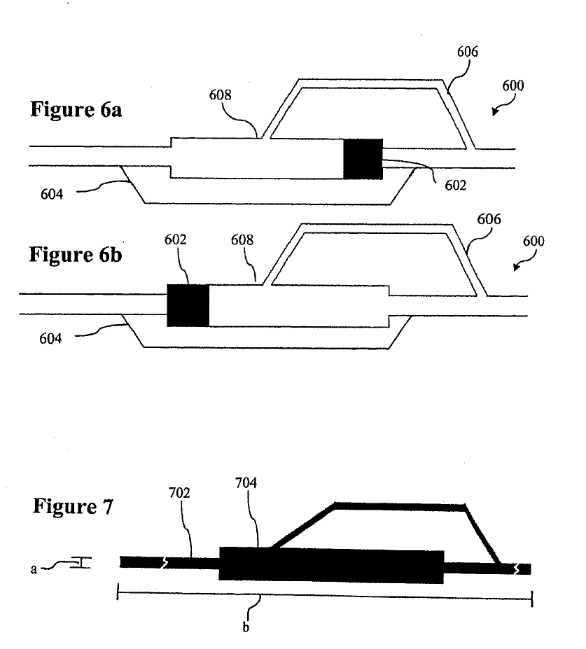

Referring now to FIGS. 6a and 6b, a device 600 includes small channel 604 and channel 606 that can selectively allow fluids to pass through a vessel while blocking larger components such as cells. In some embodiments, small channel 604 and channel 606 differ in diameter such that different size components in a fluid can pass through the different channels. In one embodiment, device 600 includes small channel 604, a movable plug 602, and a flow control channel 606. Moveable plug 602 can be similar to movable plug valve 504 described with respect to FIGS. 5a and 5b. When moveable plug 602 is in an open position, as shown in FIG. 6a, fluid may flow through channel 604, while fluid and larger cells may both flow through channel 606. Conversely, when moveable plug 602 is in a closed position, as shown in FIG. 6b, only fluid and substances less than the diameter of small channel 604 can pass through small channel 604 and cross movable plug 602. In some embodiments, small channel 604 includes a diameter less than that of a sperm cell (e.g., >20 microns) such that fluids are permitted to flow through small channel 604 of device 600 but not sperm cells when moveable plug 602 is in a closed position. This allows for hormones or other agents within the fluid to freely flow across valve 608, thus addressing such issues raised by Bucalo et al. in U.S. Pat. No. 4,013,063, which is incorporated herein by reference.

In some embodiments, small channel 604 can be less than about 5 millimeters in diameter. In some embodiments, small channel 604 can be less than about 4 millimeters in diameter. In some embodiments, small channel 604 can be less than about 3 millimeters in diameter. In some embodiments, small channel 604 can be less than about 2 millimeters in diameter. In some embodiments, small channel 604 can be less than about 1 millimeter in diameter. In some embodiments, small channel 604 can be less than about 0.5 millimeters in diameter. In some embodiments, small channel 604 can be less than about 250 micrometers in diameter. In some embodiments, small channel 604 can be less than about 100 micrometers in diameter. In some embodiments, small channel 604 can be less than about 75 micrometers in diameter. In some embodiments, small channel 604 can be less than about 50 micrometers in diameter. In some embodiments, small channel 604 can be less than about 25 micrometers in diameter. In some embodiments, small channel 604 can be less than about 15 micrometers in diameter. In some embodiments, small channel 604 can be less than about 10 micrometers in diameter. In some embodiments, small channel 604 can be less than about 5 micrometers in diameter. In some embodiments, small channel 604 can be less than about 2 micrometers in diameter.

In some embodiments, a device of the present invention may be operatively associated with a guide wire or tube for minimally invasive implantation. The valve and guide wire or tube may be configured for guiding the valve into a vessel for insertion at a desired location therein. In some embodiments, valves, or combinations of valves and guide wires or guide tubes, can be packaged together in a container in sterile form for subsequent use.

In some embodiments, the device of the present invention may be loaded with a treatment, drug, contraceptive drug, hormone, combination thereof, or the like. In some embodiments, the drug, contraceptive drug, hormone, or the like is chemically bound to or with the materials of the device. In alternative embodiments the drug, contraceptive drug, hormone, or the like is diffused from the material of the device after implantation. In some embodiments, the drug, hormone, contraceptive drug is selected from the group including, but not limited to, an antibiotic, an antiviral, an anticancer, Melatonin, androgenic hormone, progesterone, estrogen, testosterone enanthate, copper compounds, 7 a-methyl-19-nortestosterone acetate, norethindrone, or polyelectrolyte gels such as those containing ethylene vinyl acetate, maleic anhydride, hydroxyl ethyl methacrylate, poly(ethylene glycol), styrene and others.

In some embodiments, devices of the present invention may be fabricated from polymers including but not limited to: poly(dimethyl siloxane), Kratons, buna rubber, natural rubber, a fluorelastomer, chloroprene, butyl rubber, nitrile rubber, polyurethanes, hydrogels, polyelectrolytes, or other elastomeric materials and thermoplastic elastomers. In further embodiments the device may be fabricated from or coated with a material which inhibits the growth and/or adhesion of cells or tissue. In some embodiments, the materials of the device can be configured to dissolve over a predetermined period of time.

In some embodiments, the devices can be used to block the flow of other fluids or semifluids in the body including but not limited to: blood, urine, spinal fluid, pus, plueral fluid, bone marrow, saliva, mucous, sebum, sweat, tears, menses, milk, intestinal fluid, etc. The therapeutic value of the ability to control the presence and absence of such fluids using said devices is understood and incorporated herein. A specific example includes the use of such a device to selectively cut off blood supply to a tumor. The devices can be delivered to the primary blood vessels of a tumor and activated once in place to close or restrict flow of blood to or from the tumor. In some embodiments, magnetic materials within the devices can be used to guide the device to a particular site and also to actuate valves once in place.

The foregoing is illustrative of the present invention, and is not to be construed as limiting thereof. The invention is defined by the following claims, with equivalents of the claims to be included therein.

### EXAMPLES

A master template is generated on a silicon wafer using SU-8 photoresist and known photolithography techniques. The master consists of features with the structure represented in FIG. 7. The structure consists of channel 702, which has length b of 2 cm and width/height a of about 100 um. The structure contains check valve structure 704 in the middle.

A polydimethylsiloxane (PDMS) resin is then cast on to the master to a thickness of 500 microns and cured to form an elastomer. Upon separation from the master template, a molded PDMS film is generated possessing channels in the shape of the pattern on the master.

Separately, a 500 micron thick smooth film of the same PDMS resin is spin-coated on to a silicon wafer and cured to from an elastomer. Next, the patterned side of the patterned PDMS elastomer film and the surface of the smooth film are exposed to an oxygen plasma for 1 minute. The patterned surface of the PDMS film is immediately sealed to the smooth film, forming an enclosed channel structure. The two layers are left to set for 30 minutes to become bonded.

Using a controlled blade, strips of the bonded elastomer layers are cut to a width of ˜1 mm. The strips are cut such that the central 100 um channel runs through the middle of the strip. The strips are then inserted into a metal tube, exactly 1 mm in diameter. The empty portions between the strip and the diameter of the tube are then filled with PDMS resin and cured such that the strip takes the shape of the circular metal tube. Upon curing, the now cylindrical-shaped device is removed from the metal tubes. The devices are cut at both ends, opening the central channel at both ends.

Next, the channels in the device are filled with a UV curable resin containing magnetite. The fluid is cured by exposure to UV light and a photomask is placed over the device such that only a small region of the magnetic fluid is cured within the check valve structure. After curing, the channels are flushed to remove the uncured liquid resin and leave the desired cured plug in place. This plug can be magnetically addressed to open and close the check valve as shown in FIGS. 5a and 5b.

1-46. (canceled)

47. A medical device, comprising:
a body portion configured and dimensioned to be associated with a vessel of a patient; and
a responsive component associated with the body portion wherein the responsive component is switchable between a first configuration and a second configuration.

48. The medical device of claim 47, wherein the first configuration restricts flow through the vessel and the second configuration does not restrict flow through the vessel.

49. The medical device of claim 47, wherein the body portion is configured and dimensioned to receive an external surface of the vessel.

50. The medical device of claim 47, wherein the body portion is configured and dimensioned to be positioned within the vessel.

51. The medical device of claim 47, wherein the vessel is selected from the group consisting of a vas deferens, a fallopian tube, a urethra, and a ureter.

52. The medical device of claim 47, further comprising a controller configured to manipulate the responsive component between the first configuration and the second configuration.

53. The medical device of claim 47, wherein the responsive component is an electrostrictive, magnetostrictive, piezoelectric actuator, or shape-memory material.

54. The medical device of claim 47, wherein the responsive component comprises at least one ferromagnetic particle.

55. The medical device of claim 47, further comprising an indicator operatively associated with the responsive component for indicating whether the responsive component is in the first configuration or the second configuration.

56. The medical device of claim 47, wherein the body portion is substantially cylindrical in shape, and wherein the body portion has an elongate slit formed axially therethrough configured and dimensioned to receive the vessel.

57. The medical device of claim 47, wherein the body portion comprises a material that inhibits tissue growth or adhesion of substances.

58. The medical device of claim 47, wherein the body portion comprises perfluoropolyether.

59. A medical device, comprising:
a body portion configured and dimensioned to be positioned in a vessel of a subject without cutting through the vessel; a channel defined in the body portion through which the vessel can pass; and
a responsive component associated with the body portion for reversibly closing the channel such that fluid in the vessel is restricted from flowing through the vessel.

60. The medical device of claim 59, wherein the vessel is selected from the group consisting of a vas deferens, a fallopian tube, a urethra, a ureter, a duct, intestine, an artery, and vein.

61. The medical device of claim 59, wherein the responsive component is selected from the group consisting of electrostrictive, magnetostrictive, piezoelectric, shape-memory polymer, and ferromagnetic particle components.

62. The medical device of claim 59, further comprising an indicator operatively associated with the responsive component for indicating an open or closed position of the responsive component.

63. A method of controlling flow through a vessel of a patient, comprising:
associating a medical device with a vessel of a patient wherein the medical device comprises: a body portion configured and dimensioned to be associated with the vessel of the patient; a responsive component associated with the body portion for reversibly restricting flow through the vessel; and
controlling the responsive component to restrict or allow flow through the vessel.

64. The method of claim 63, wherein associating the medical device with a vessel comprises implanting the medical device within the vessel.

65. The method of claim 63, wherein associating the medical device with the vessel comprises associating the body portion with an outer diameter of the vessel.
